(19) United States
(12) Patent Application Publication
(10) Pub. No.: US 2009/0299137 A1
(43) Pub. Date: Dec. 3, 2009

(54) ABDOMINAL OBSERVATION DEVICE

(75) Inventors: Ehud Gal, Reut (IL); Zahi Ben Aroya, Gealia (IL); Gennadiy Berinsky, Modi'in (IL)

Correspondence Address: MERCHANT & GOULD PC, P.O. BOX 2903, MINNEAPOLIS, MN, 55402-0903, US

(73) Assignees: Wave Group Ltd., Tel Aviv (IL); O.D.F. Medical Ltd, Tel Aviv (IL)

(21) Appl. No.: 12/302,457

(22) PCT Filed: May 3, 2007
(86) PCT No.: PCT/IL07/00540

§ 371 Date: Aug. 10, 2009

(30) Foreign Application Priority Data
May 31, 2006 (IL) 176046

(51) Int. Cl. A61B 1/04

(52) U.S. Cl. 600/109; 600/116

(57) ABSTRACT

A medical imaging device enables observation within the abdominal and pelvic cavity at a wide field of view and enables medical procedures to be carried out within the wide field of view. The medical imaging device includes a central assembly and a sterile disposable cover, which is slipped over the central assembly. The central assembly has a proximal portion, which remains outside of the abdominal cavity of the patient, and a distal portion, which is inserted into the abdominal cavity through a slit made in the abdominal wall by the surgeon. A first plurality of light emitting diodes is circumferentially distributed around the outer surface of the central assembly near the top of the distal portion and a reversibly inflatable balloon is circumferentially attached to the cover at the location of the first plurality of diodes. When the balloon is inflated and the first array of light emitting diodes is activated to produce light, then the light enters the interior of the balloon, is repeatedly reflected from the inner walls of the balloon until it eventually passes through the wall of the balloon at random angles thereby illuminating the entire interior of the abdominal and pelvic cavities. Also, a system includes the medical imaging device, an observation unit comprising a controller, a processor, and display; and a communicator between the medical imaging device and the observation unit.

### FIELD OF THE INVENTION

The invention is in the field of medical devices. More specifically the invention refers to the fields of laparoscopy.

### BACKGROUND OF THE INVENTION

Endoscopes are devices which enable penetration into a patient's body for observation, diagnosis or surgery. Endoscopes which are used for surgery inside the abdominal and pelvic cavity area are called “Laparoscopes”. In order to perform these functions laparoscopes must be equipped to enable several tasks to be carried out. These tasks include: illumination, observation, and imaging of the area of interest; insufflation of the abdominal cavity with gas to enable the surgeon convenient operational space and access to the operation area during surgery; insertion of operational surgery tools; and taking tissues samples.

Traditional laparoscopic procedures frequently require the use of two or more trocars to carry out these tasks, which often means that one or more assistants must work in coordination with the surgeon to perform the procedure. Restricted vision, difficulty in handling of the instruments (hand-eye coordination), lack of tactile perception, and limited working area are factors which add to the technical complexity of this surgical approach.

Many of the difficulties of the prior art could be eliminated by use of an omni-view laparoscope, which is an instrument that provides the surgeon with the capabilities of observation of the operated area as well as it's surroundings. This device could reduce the time needed to carry out the procedure, facilitate the work of the surgeon, possibly remove the necessity of working with an assistant, and reduce the risk and complications that accompanies the penetration of the abdominal cavity at several locations in order to insert all of the tools needed to perform the procedure. The design of an omni-view laparoscope requires the integration of several elements including:
miniature optics and an imaging device to enable imaging at the desirable resolution not only of the area directly in front of the distal tip of the laparoscope, but with a wide field of view (FOV) to also show the surroundings;
a miniature illumination assembly to supply homogenous illumination to the entire FOV;
a miniature cooling assembly to remove the heat resulting from the operation of the illumination assembly, the imaging device, and any additional electronics within the device;
working channels to enable the delivery of sterile surgical tools to the operation area through the device;
channels to allow introduction of fluids and/or, e.g. to introduce gas for insufflation of the abdominal cavity gas and/or suction;
   all of these components must be “packaged” in a device that can be easily sterilized and whose dimensions are compatible with its function.

A purpose of the invention is to provide a medical device which enables a wide FOV observation inside the abdominal and pelvic cavities area.

Another purpose of the invention is to provide a medical device which enables multi use and sterility in a simple manner.

Another purpose of the invention is to provide a medical device which enables illumination of areas of the abdominal and pelvic cavities surrounding the device.

Another purpose of the invention is to provide a medical device which enables cooling capabilities.

Another purpose of the invention is to provide a medical device which enables insufflation of the abdominal cavity.

Another purpose of the invention is to provide a system using the medical device that enables processing of the acquired information and presentation of the information in an intuitive manner to the observer.

Another purpose of the invention is to provide a system using the medical device that enables “hand free” control over the field of view and its parameters.

Another purpose of the invention is to provide a system using the medical device that enables integration of software to analyze the information acquired and to enable automatic/semi-automatic detection of abnormalities.

Another purpose of the invention is to provide a system using the medical device which enables cooperation with additional imaging systems in order to enable three dimensional imaging of the abdominal and pelvic cavities.

Another purpose of the invention is to provide a system using the medical device which enables advanced solutions to medical problems including observation as well as treatment.

Another purpose of the device is to enable full documentation of procedures and diagnosis made during the operation.

Another purpose of the device is to enable a safe transmission of the FOV for remote consulting (telemedicine).

Further purposes and advantages of this invention will appear as the description proceeds.

### SUMMARY OF THE INVENTION

In a first aspect the invention is a medical imaging device that enables observation within the abdominal and pelvic cavity at a wide field of view and enables medical procedures to be carried out within the wide field of view. The medical imaging device of the invention comprises:
a. a central assembly comprised of a proximal portion, which remains outside of the abdominal cavity of the patient, and a distal portion, which is inserted into the abdominal cavity through a slit made in the abdominal wall by the surgeon; and
b. a sterile disposable cover, which is slipped over the central assembly.

The medical imaging device of the invention is characterized in that a first plurality of light emitting diodes is circumferentially distributed around the outer surface of the central assembly near the top of the distal portion and a reversibly inflatable balloon is circumferentially attached to the cover at the location of the first plurality of diodes. When the balloon is inflated and the first array of light emitting diodes is activated to produce light, then the light enters the interior of the balloon, is repeatedly reflected from the inner walls of the balloon until it eventually passes through the wall of the balloon at random angles thereby illuminating the entire interior of the abdominal and pelvic cavities.

The balloon is preferably configured such that when inflated it stabilizes the device and seals the slit in the abdominal wall from within.

The imaging is accomplished by means of a wide field of view lens and an electronic camera mounted on the distal tip of the device. In preferred embodiments of the invention the wide field of view lens is an omni-directional lens and the device comprises a second plurality of light emitting diodes located on the distal tip of the central assembly to provide forward illumination for the camera.

The cover is designed to be easily removable from the central assembly and discarded after the procedure, thereby minimizing the sterilization process of the central assembly. The disposable cover may comprise one or more channels that are used to fulfill on or more of the following functions:
a. introduction of gas to inflate the balloons and alternately to draw out the air allowing the balloons to be deflated;
b. introduction of gas for insufflation of the abdominal cavity;
c. introduction of gas to assist in maintaining the window clear of fluids and other substances which might accumulate on the window and obscure the FOV during the procedure and to aid in cooling the device;
d. insertion of operating tools to be used by the surgeon as required;
e. introduction of cool gas for dissipating the heat generated by the LEDs; and
f. provision of a way for allowing the cool gas to escape, thereby transporting the heat away from the interior of central assembly.

Embodiments of the medical imaging device of the invention comprise an anchoring device, which rests on the exterior of abdominal wall. The anchoring device may comprise one or more of: means for creating a seal between it and the abdominal wall, means for adjusting the depth to which the distal portion is inserted into the abdominal cavity, or means for locking the device at the desired depth. The anchoring device may also comprise a ball joint through which the distal portion of the central assembly passes as it is inserted into the abdominal cavity.

The medical imaging device of the invention may comprise a second reversibly inflatable balloon placed on the proximal portion of the device adjacent to the abdominal wall. In preferred embodiments the anchoring device is replaced by a second reversibly inflatable balloon.

Embodiments of the medical imaging device comprising channels in the cover comprise a manifold mounted on the proximal portion of the device to control the pressure and flow rates of gases through the channels.

Embodiments of the medical imaging device comprise one or more mechanical joints or an articulation section in the distal portion of the central assembly that can be activated by the surgeon from a control handle provided on the proximal portion.

Preferably the central assembly can be rotated about its longitudinal axis either manually or by means of a small motor attached to or located within the anchoring device.

In another aspect the invention is a system that enables observation within the abdominal and pelvic cavities at a wide field of view and enables medical procedures to be carried out within the wide field of view. The system comprises:
a. a medical imaging device of the invention;
b. an observation unit comprising control means, processing means, and display means; and
c. means for communicating between the medical imaging device and the observation unit.

Embodiments of the processing means comprise one or more of: software for processing the video images, means for combining individual images to produce and display high quality 360 degree images of the abdominal and pelvic cavities, or memory means. The processing means of the observation unit can enable stabilization of the acquired imagery information.

Some or all of the components of the system can be controlled either manually or automatically by means of software contained in the processing means.

The observation unit can be adapted to allow the operator to control one or more of the following parameters: the camera frame rate, the camera resolution, the camera focus, the camera zoom on selected regions of interest, the level of illumination of the cavity, the pressure and flow rate of gases, and pressure in the abdominal and/or pelvic cavities.

The observation unit can be adapted to allow the operator to select the display mode which allows him to present the imagery information gathered by the imaging device in the manner that is most clear to him.

Preferably the observation unit is adapted to enable performance of medical image understanding (MIU) of the acquired information. The MIU capabilities can be used for automatic detection of abnormalities of tissues during surgery using an algorithm which enables analysis of the acquired images. The MIU capabilities can be used to automatically detect abnormalities such as hemorrhages or operating tools accidentally left inside the cavity during the surgery process.

Embodiments of the observation unit are adapted to enable interface with additional medical imaging systems that might be present. In these embodiments the interface enables three dimensional displays of the abdominal and pelvic cavities, based on all the information available from multiple sources.

All the above and other characteristics and advantages of the invention will be further understood through the following illustrative and non-limitative description of preferred embodiments thereof, with reference to the appended drawings.

### BRIEF DESCRIPTION OF THE DRAWINGS

FIG. 1 is cross-sectional side view schematically showing the distal portion of the central assembly covered by disposable cover;

FIG. 2 is a view along plane A-A of FIG. 1;

FIG. 3 schematically shows the distal portion of central assembly surrounded by disposable cover inserted through abdominal wall into the abdominal cavity;

FIG. 4 schematically shows one embodiment of the entire system of the invention;

FIG. 5 and FIG. 6 schematically show embodiments of the invention which allow control of the observation direction inside the abdominal and pelvic cavities; and

FIG. 7 schematically shows another embodiment of the entire system of the invention.

### DETAILED DESCRIPTION OF PREFERRED EMBODIMENTS

The descriptions herein are schematic descriptions of possible embodiments of the present invention. Although the entire system is comprised of several different components, reference to only the main components is made for the sake of brevity since the existence of the minor components and their incorporation into the system are well within the knowledge of those skilled in the art. The figures are designed merely to provide a general perception of preferred embodiments of the present invention. It is emphasized that lack of reference to the entire variety of integration and production of the possible embodiments shall not impose a restriction over the extent of the invention hereof.

The present invention is a unique medical device which enables wide Field Of View (hereinafter: “FOV”) observation during surgeries which are performed inside the abdominal and pelvic cavity and a system for performing laparoscopic procedures comprising the device. The use of wide FOV observation enables the surgeon to better orient himself within the operation area and improves the detection of tissue irregularities and other abnormalities. Wide FOV also enables early detection of tissue and organ damage caused during the operation. In addition, the information acquired by the wide FOV observation device can be processed and presented using an interface on the observation unit, and integral intelligent software can analyze the information and enable automatic detection of tissue irregularities and other abnormalities. Relevant information can be documented efficiently for later use, e.g. during debriefing or consultations.

The medical device of the present invention comprises optics which enables a wide FOV which is wider than the standard laparoscopic view, which is typically up to 45 degrees horizontally and vertically, and provides what is known as an “Omni-Directional” FOV, i.e. up to an almost full 360 degrees around the axis of the device.

The special optics capable of enabling wide FOV as well as Omni-Directional FOV are well known in the art including commercially available optics such as “Fish Eye” lenses, which provide up to about 180 degree FOV and are also known as “half sphere” FOV. In addition, some advanced optical lenses enable observation at a selected preplanned FOV almost up to 360 degrees. Some of the advanced lenses enable in addition zoom capabilities at predefined Regions of Interest (ROI) within the selected FOV. The information acquired by the device can be displayed on a computer screen using special software and a Man Machine Interface (MMI) in order to assist the observer to orient himself in the wide FOV images.

Typical methods of applying Omni-directional imaging using special optics and methods are described in the following publications:
U.S. Pat. No. 6,304,285 to Geng—The invention is an imaging system comprising a mirror for viewing objects within a hemispherical FOV, a projector for projecting light towards the mirror, a variable wavelength filter between the projector and the mirror, a detector, and a image generator to generate three-dimensional images of the objects viewed by the mirror.
U.S. Pat. No. 5,790,182 to St. Hilaire—The invention is a system providing a wide field of view comprised of two mirrors concentrically positioned relative to one another. The radii of the two mirrors are related by the square of the “golden ratio” to reduce aberrations.
WO 02/059676 to Gal, et al.—Describes lenses with asymmetric convex surfaces enabling a wide FOV.
WO 03/026272 to Gal, et al.—Describes lenses comprising both asymmetric and symmetric surfaces.
WO 02/075348 to Gal, et al.—The invention is a method of detection of the bearing and elevation of radiation sources using an Omni-directional lens.
WO 04/042428 to Gal, et al.—Describes lenses that enable acquiring an Omni-directional FOV and in addition comprise means to simultaneously provide Omni-directional illumination of the FOV of the lens.
WO 04/008185 to Gal, et al.—Describes a wide-angle imaging assembly which comprises a main lens produced from a single aspheric optical block.

Many additional methods of applying wide FOV optics to imaging systems are known. It is emphasized that the exact design of the optical system is not a critical feature of the present invention. Embodiments of the system can easily be produced that use any optical system, either known from the prior art or novel, that is capable of providing a wide angle, preferably omni-directional FOV.

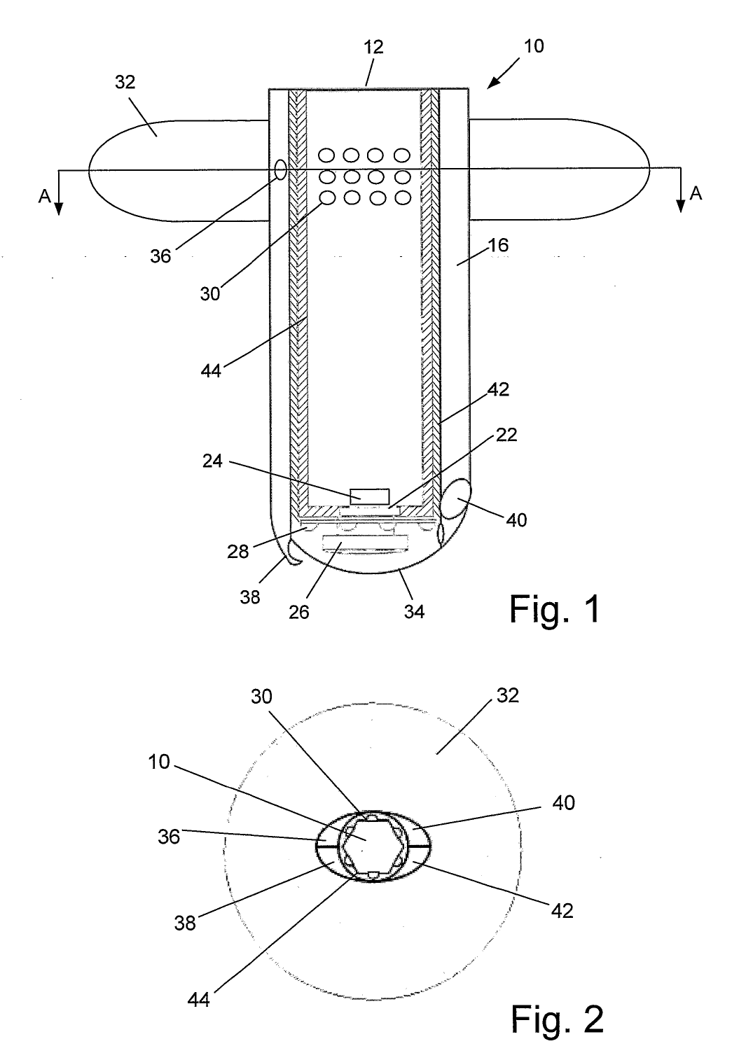

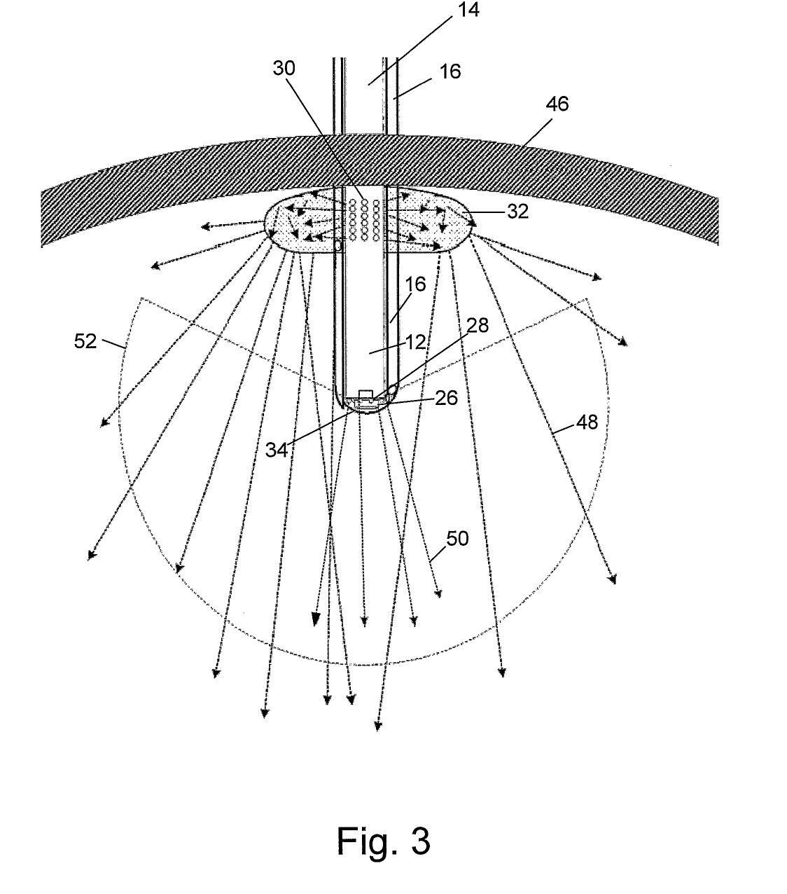

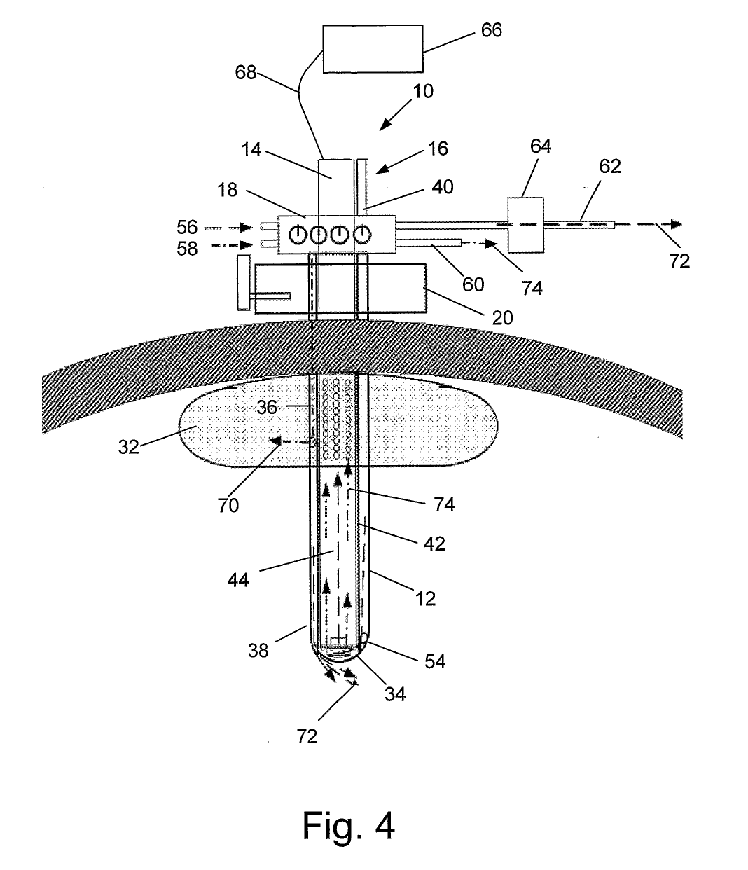

The laparoscope of the invention comprises a central assembly and a sterile disposable cover. The system of the invention comprises a laparoscope of the invention and an observation unit. The central assembly comprises a proximal portion, which remains outside of the abdominal cavity of the patient, and a distal portion, which is inserted into the abdominal cavity through a slit made in the abdominal wall by the surgeon. The proximal portion of the central assembly will be described hereinbelow with respect to FIG. 4 and the distal portion with respect to FIG. 1 and FIG. 2. The device can be inserted into the abdominal cavity from any angle and used to perform any laparoscopic procedure.

The disposable cover slips over the central assembly and isolates the central assembly from direct contact with body tissue and fluids. The cover contains several channels whose purpose will be described hereinbelow. In addition a reversibly inflatable balloon is circumferentially attached to the outside of the cover at the upper end of the distal portion of the central assembly. The balloon is used to aid in illuminating the abdominal and pelvic cavities. The balloon also can be configured such that when inflated it stabilizes the device and seals the slit in the abdominal wall from within for purposes of sterilization and to allow insufflation of the abdominal and pelvic cavities. After the procedure, the cover can be easily removed from the central assembly and discarded, thus minimizing the sterilization process of the central assembly.

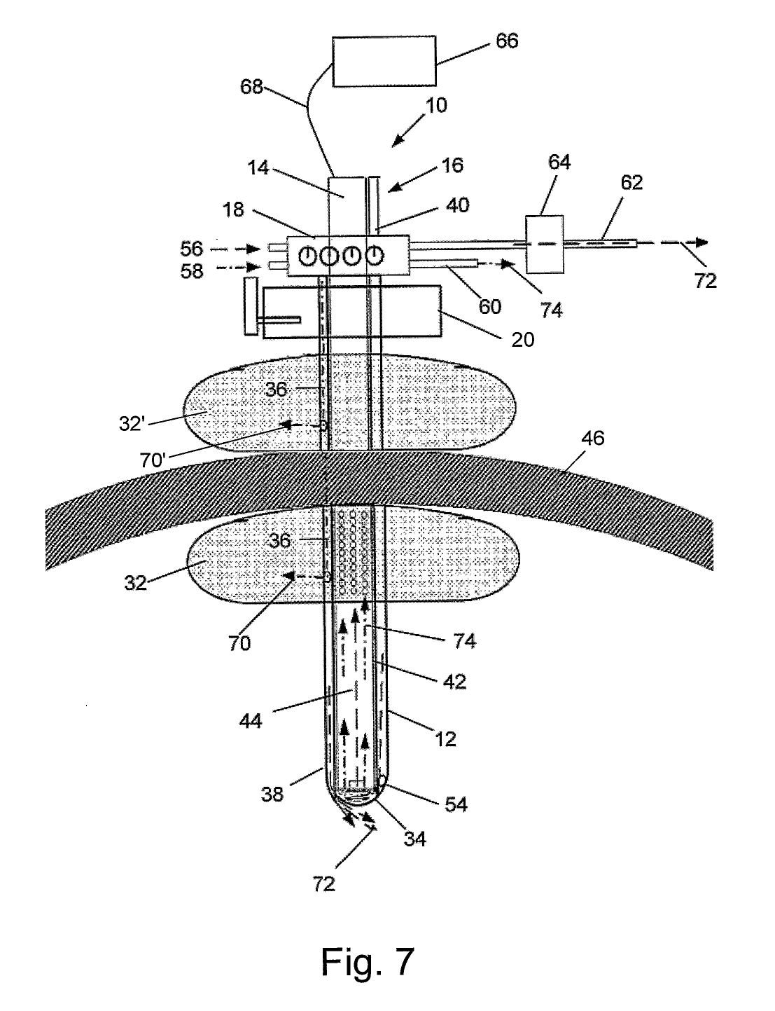

In preferred embodiments a second balloon (shown in FIG. 7) is placed on the proximal portion of the device adjacent to the abdominal wall for better sealing of the abdominal slit and for fixation and stabilization of the device.

FIG. 1 is cross-sectional side view schematically showing the distal portion 12 of the central assembly 10 covered by disposable cover 16. FIG. 2 is a cross-sectional view taken along plane A-A in FIG. 1. The central assembly 10 comprises an imaging device, i.e. camera comprised of a sensor 22, e.g. a CCD sensor, and an electronic driver, which is connected to the observation unit by means of cables (not shown in the figures); a wide field of view lens 26, e.g. a standard commercial lens enabling a FOV of approximately 180 degrees; a first plurality of LEDs 30 circumferentially distributed on the outer surface of the central assembly near the top of distal portion 12 to provide illumination to the interior of the abdominal and pelvic cavities as will be described with reference to FIG. 3, and a second plurality of light emitting diodes (LEDs) 28 located on the distal tip of central assembly 10 around lens 26 to provide forward illumination for the camera.

Cover 16 is shown surrounding central assembly 10 in FIG. 1 and in FIG. 2, which is a cross sectional view along plane A-A in FIG. 1. Balloon 32, which is attached to cover 16, is inserted into the abdominal cavity in a deflated state and once in position inside the abdominal cavity is inflated using gas, e.g. CO₂ or air, which is introduced through channel 36 in the cover. In the embodiment shown, LEDS 30 are mounted either on the outside of central assembly 10 or so that they project through the wall of central assembly 10. In this case, the walls of the cover 16 are transparent to the wavelengths of light emitted by LEDs 30 at least in the areas overlying the LEDs. In an alternate embodiment, LEDs are located inside of central assembly 10 and windows are provided to allow the light to be transmitted through the walls of central assembly 10 and the cover. In all cases the light emitted by LEDs 30 passes through cover 16 into the interior of inflated balloon 32. Balloon 32 is semi transparent to the wavelengths of the light emitted by LEDs 30.

FIG. 3 schematically shows the distal portion 12 of central assembly 10 surrounded by disposable cover 16 inserted through abdominal wall 46 into the abdominal cavity. The balloon 32 has been inflated and light from LEDs 30, which enters the interior of the balloon, is repeatedly reflected from the inner walls of balloon 32 until it eventually passes through the semi transparent wall of the balloon at random angles as shown schematically by dotted arrows 48. The result is that the balloon 32 essentially functions as a lamp which illuminates the entire interior of the abdominal and pelvic cavities.

The light from the balloon 32 illuminates all of the FOV (the area inside dotted line 52 in FIG. 3) of lens 26. To obtain a bright clear image of the area directly in front of the camera, which is normally the area where the medical procedure is performed, and also to compensate for blockage of part of the light by the instrument itself a plurality of LEDs 28 is provided on the distal end of the central assembly 10. Light from LEDs 28, schematically shown in FIG. 3 as dashed arrows 50, is transmitted through optically transparent window 34 on the distal end of disposable cover 16. LEDs 28 and 30 produce light in the wavelength range that can be detected by sensor 22. The materials and different degrees of transparency of which the windows in the central assembly, cover, and balloon are manufactured are chosen to have properties to optimize the distribution of light within the abdominal and pelvic cavities and can be changed to provide optimal illumination for different procedures.

The disposable cover 16 comprises a number of channels that are used to fulfill various functions necessary for performing the medical procedure. Shown in FIG. 1 and FIG. 2 are the following channels:
Channel 36 is used to introduce gas to inflate the balloon 32 and alternately can be used to draw out the air allowing the balloon to be deflated so that the instrument can be withdrawn from the abdominal cavity.
Channel 38 is used to introduce gas for insufflation of the abdominal cavity. In the embodiment shown in the figures the gas which inflates the abdominal cavity is released into the abdominal cavity and directed such that it flows over transparent window 34 at the distal tip of disposable cover 16. In this way the flowing gas assists in maintaining the window clear of fluids and other substances which might accumulate on the window and obscure the FOV during the procedure. The gas flow can also aid in cooling the unit.
Channel 40 is used to enable insertion of operating tools to be used by the surgeon as required. This is an optional channel, the inclusion of which depends on the procedure to be performed using the device.
Channel 42 is used to introduce cool gas for dissipating the heat generated by the LEDs.
Channel 44 is used to allow the gas introduced through channel 42 to escape, thereby transporting the heat away from the interior of central assembly 10.

In order to enable continuous operation within the abdominal cavity, a cooling assembly is provided to prevent the heat generated by the LEDs and electronics in the central assembly from raising the temperature inside the abdominal cavity above an acceptable value. In the embodiment shown, the cooling assembly is based on circulation of cooled gas, e.g. compressed air or CO₂. The gas is caused to flow from an external source through channel 42 to the distal end of the central assembly and back out of the device through channels 44 absorbing heat on the way and thus maintaining the system at an acceptable temperature during the procedure. Channels 44 can be located in the interior of central assembly 10 or around the outside wall of the central assembly in thermal contact such that the gas flowing through them will absorb the heat generated in the interior. In the embodiment shown in FIG. 2, central assembly 10 has a hexagonal cross section and the LEDs 30 project out of the wall of the central assembly thereby creating space between the outer wall of central assembly 10 and the inner wall of cover 16 that act as channels 44, allowing free flow of the cooling gas and efficient cooling. In other embodiments cooling means, such as a thermoelectric cooler can be incorporated into the device.

FIG. 4 schematically shows one embodiment of the entire system of the invention. The proximal portion 14 of central assembly 10 and of the disposable cover 16 remain outside of the abdominal cavity and distal portion 12 of central assembly 10 surrounded by the distal portion of cover 16 is shown inserted into the abdominal cavity through a slit cut by the surgeon in the abdominal wall 46. The device is stabilized by inflated balloon 32 inside the abdominal cavity and anchoring device 20, which rests on the exterior of abdominal wall 46. Anchoring device 20 may comprise means for creating a seal between it and the abdominal wall to limit motion and prevent the flow of fluids or other matter into or out of the abdominal cavity. Additionally anchoring device 20 comprises means for adjusting the depth to which the distal portion 12 is inserted into the abdominal cavity and for locking the device at the desired depth.

A manifold 18, comprising a number of valves is mounted on the proximal portion 14 above anchoring device 20. The manifold 18 enables control over the supply of gas from external sources, e.g. a hospital central distribution network, through the channels described herein in the cover of the device. For example CO2 is lead through tubing 56 and a control valve into channel 36 leading to balloon 32. The control valve on manifold enables control of the pressure of the CO2 in the balloon and also on the direction of the flow thus enabling deflation of the balloon after the usage in order to allow smooth extraction of the device from the abdominal. The CO2 also flows through tubing 56 and another control valve on manifold 18 into channel 38 for insufflation of the abdominal cavity and to keep window 34 clean during the procedure. The CO₂ used for insufflation can be allowed to escape from the abdominal cavity through filter 64 and tube 62. Gas for cooling, e.g. compressed air, is introduced from an external source through tubing 58 and a valve to control the flow rate on manifold 18 into channel 42. The air absorbs heat as it flows through channels 44. Channels 44 are connected in the manifold 18 to exit tube 60, which allows the heated air to escape from the device. In FIG. 4, arrows 70, 72, and 74 show the direction of flow of the gas used to inflate balloon 32, insufflation, and cooling respectively.

In FIG. 4 are also shown the working channel 40 for the insertion of tools used during the procedure and observation unit 66. Observation unit 66 is shown connected to the central assembly by cable 68, which conducts video signals from the camera; electrical energy and control signals to the camera and LEDs; and control signals to the elements of the device. There are various methods of providing the communication between the device and the observation unit. the communication can be wired as depicted in FIG. 4, or preferably can be wireless. The information transmitted can be in many forms, e.g. digital or analog, compressed, decoded, multiplexed or raw.

Observation unit 66 comprises a control means, processing means, and display means. The processing means include software for processing the video images and memory means to enable documentation of any acquired information.

The various components of the system can be controlled either manually by the control means or automatically by means of software contained in the processing means. The observation unit 65 provides what is referred to as a man-machine interface (MMI). The MMI allows the operator to select the display mode which allows him to present the wide FOV imagery information gathered by the imaging device in the manner that is most clear to him. In a preferred embodiment, the system's MMI enables control over the central assembly's components. Thus enabling certain capabilities such as; control over the camera frame rate or resolution, focus and zoom on certain regions of interest, increase or decrease the illumination of the cavity as required, and physical parameters such as the pressure and flow rate of gases, pressure in the abdominal cavity.

In preferred embodiments, the processor of the observation unit enables stabilization of the acquired imagery information. The stabilization capability improves the displayed video by eliminating trembling of the captured video caused by motion of the device inside the abdominal cavity.

The MMI preferably enables performance of medical image understanding (MIU) of the acquired information. The MIU capabilities can be used for example for automatic detection of abnormalities of tissues during surgery using an algorithm which enables analysis of the acquired images. In addition, the MIU capabilities can also automatically detect abnormalities during the surgery process such as hemorrhages, operating tools accidentally left inside the cavity during surgery, etc. Additionally, the observation unit preferably enables interface with additional medical imaging systems that might be present. In a preferred embodiment, the interface enables three dimensional presentations of the abdominal and pelvic cavities, based on all the information available from multiple sources (this capability is sometimes known as “sensor fusion”). Preferred embodiments of the invention comprise memory means to enable full documentation of procedures and diagnosis made during the procedure and means to enable safe transmission of the FOV for remote consulting (telemedicine). As a result of these capabilities, the system enables advanced solutions to medical problems including observation as well as treatment.

After the procedure has been completed the air is removed from balloon 32, for example by means of a vacuum pump attached to tubing 62, and the distal portion 12 is withdrawn from the abdominal cavity. In some embodiments the cover 16 has a recess into which the deflated balloon fits, thereby making the device easier to insert and withdraw from the abdominal cavity. Once the device is withdrawn, the slit in the abdominal wall 46 is closed, e.g. with surgical clips or stitches, the cover 16 is removed from the central assembly 10 and discarded, a new cover 16 is slipped over central assembly 10, and the device is ready for use with another patient.

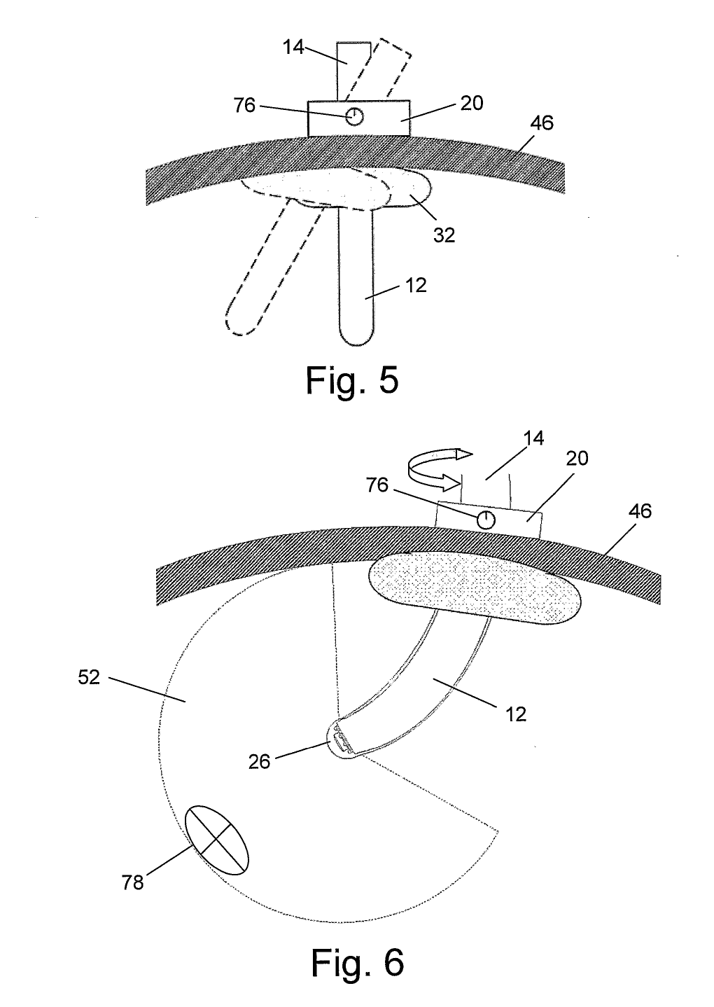

FIG. 5 and FIG. 6 schematically show embodiments of the invention which allow control of the observation direction inside the abdominal cavity. In the embodiment shown in FIG. 5, anchoring device 20 comprises a ball joint (not shown) through which the distal portion 12 of the central assembly passes as it is inserted into the abdominal cavity. The assembly is inserted generally perpendicular to the abdominal wall 46 and the balloon 32 is inflated (solid lines in FIG. 5). Now the surgeon tilts the device by pushing on the proximal portion 14 of the central assembly 14. He can also slide the device back and forth through the ball joint until the images on the display of the observation unit are optimized for viewing the area at which the procedure is to be carried out. Once the optimal orientation of the device (dotted lines in FIG. 5) has been achieved, locking mechanism 76 is tightened preventing further rotational or lateral motion of the device. The inflated balloon 32 has no difficulty in adapting itself to the changing orientation of the central assembly, therefore optimal illumination of the abdominal and pelvic cavities is always provided.

In FIG. 6 is shown an embodiment in which the distal portion 12 of the central assembly comprises one or more mechanical joints or an articulation section that can be activated by the surgeon from a control handle provided on the proximal portion 14. The central assembly can also be rotated as shown by arrow 78 either manually or with the aid of a small motor attached to or located within anchoring device. In this way the surgeon can point the center of the field of view 52 of lens 26 directly at the area 78 at which the procedure is to be carried out. Once the camera is optimally oriented, locking mechanism 76 is tightened preventing further motion of the device. If the articulation section is locked and locking mechanism 76 is tightened to prevent lateral motion, however the device can still be rotated, than high quality images can be obtained of the entire abdominal and pelvic cavities. In cases where the lens is not an omni-directional lens, the image processing program in the processor of the observation unit can combine individual images to produce and display high quality 360 degree images of the abdominal and pelvic cavities

FIG. 7 schematically shows a preferred embodiment of the entire system of the invention. In this embodiment a second balloon 32′ is placed on the proximal portion of the device adjacent to the abdominal wall 46 and below anchoring device 20. In some embodiments comprising the second balloon, the anchoring device 20 is not included and balloon 32′ is located between manifold 18 and the abdominal wall 46. Balloon 32′ is adapted to seal the abdominal slit from the outside and to hold the device in place and stabilize it on the body of the patient. Arrow 70′ schematically shows air entering balloon 70′. The external balloon can be inflated and deflated through channel 36 in the same manner as the internal balloon. Alternatively balloon 32′ can be directly connected to a source of compressed air and vented either through a separate channel in device 10 or by means external to device 10. External balloon 32′ may be the same as internal balloon 32, but it can be different and in particular it does not have to have the same optical transparency as the internal balloon. With the exception of the additional balloon 32′, all other components of the system that are shown in FIG. 8 are identical to those in the embodiment shown in FIG. 4.

Although embodiments of the invention have been described by way of illustration, it will be understood that the invention may be carried out with many variations, modifications, and adaptations, without exceeding the scope of the claims.

1. A medical imaging device enabling observation within the abdominal and pelvic cavity at a wide field of view and enabling medical procedures to be carried out within said wide field of view, said medical imaging device comprising:
a. a central assembly comprised of a proximal portion, which remains outside of the abdominal cavity of the patient, and a distal portion, which is inserted into said abdominal cavity through a slit made in the abdominal wall by the surgeon; and
b. a sterile disposable cover, which is slipped over said central assembly; wherein a first plurality of light emitting diodes is circumferentially distributed around the outer surface of said central assembly near the top of said distal portion, a reversibly inflatable balloon is circumferentially attached to said cover at the location of said first plurality of diodes, and when said balloon is inflated and said first array of light emitting diodes is activated to produce light, then said light enters the interior of said balloon, is repeatedly reflected from the inner walls of said balloon until it eventually passes through the wall of said balloon at random angles thereby illuminating the entire interior of said abdominal cavity, as well as said pelvic cavity.

2. A medical imaging device according to claim 1, wherein the balloon is configured such that when inflated it stabilizes the device and seals the slit in the abdominal wall from within.

3. A medical imaging device according to claim 1, wherein the cover is designed to be easily removable from the central assembly and discarded after the procedure, thereby minimizing the sterilization process of said central assembly.

4. A medical imaging device according to claim 1, wherein the imaging is accomplished by means of a wide field of view lens and an electronic camera mounted on the distal tip of said device.

5. A medical imaging device according to claim 4, wherein the wide field of view lens is an omni-directional lens.

6. A medical imaging device according to claim 4, comprising a second plurality of light emitting diodes located on the distal tip of the central assembly to provide forward illumination for the camera.

7. A medical imaging device according to claim 1, wherein the disposable cover comprises one or more channels that are used to fulfill on or more of the following functions:
a. introduction of gas to inflate the balloons and alternately to draw out the air allowing said balloons to be deflated;
b. introduction of gas for insufflation of the abdominal cavity;
c. introduction of gas to assist in maintaining the window clear of fluids and other substances which might accumulate on the window and obscure the FOV during the procedure and to aid in cooling said device;
d. insertion of operating tools to be used by the surgeon as required;
e. introduction of cool gas for dissipating the heat generated by the LEDs; and
f. provision of a way for allowing said cool gas to escape, thereby transporting the heat away from the interior of central assembly.

8. A medical imaging device according to claim 1, comprising an anchoring device, which rests on the exterior of abdominal wall.

9. A medical imaging device according to claim 8, wherein the anchoring device comprises means for creating a seal between it and the abdominal wall.

10. A medical imaging device according to claim 8, wherein the anchoring device comprises means for adjusting the depth to which the distal portion is inserted into the abdominal cavity;

11. A medical imaging device according to claim 10, wherein the anchoring device comprises means for locking said device at the desired depth.

12. A medical imaging device according to claim 1, comprising a second reversibly inflatable balloon placed on the proximal portion of said device adjacent to the abdominal wall.

13. A medical imaging device according to claim 8, wherein the anchoring device is replaced by a second reversibly inflatable balloon.

14. A medical imaging device according to claim 7, comprising a manifold mounted on the proximal portion of said device to control the pressure and flow rates of gases through the channels.

15. A medical imaging device according to claim 8, wherein the anchoring device comprises a ball joint through which the distal portion of the central assembly passes as it is inserted into the abdominal cavity.

16. A medical imaging device according to claim 1, wherein the distal portion of the central assembly comprises one or more mechanical joints or an articulation section that can be activated by the surgeon from a control handle provided on the proximal portion.

17. A medical imaging device according to claim 1, wherein the central assembly can be manually rotated about its longitudinal axis.

18. A medical imaging device according to claim 1, wherein the central assembly can be rotated about its longitudinal axis by means of a small motor attached to or located within the anchoring device.

19. A system enabling observation within the abdominal and pelvic cavities at a wide field of view and enabling medical procedures to be carried out within said wide field of view, said system comprising:
a. a medical imaging device according to claim 1;
b. an observation unit comprising control means, processing means, and display means; and
c. means for communicating between said medical imaging device and said observation unit.

20. A system according to claim 19, wherein the processing means comprises software for processing the video images.

21. A system according to claim 20, wherein, in cases where the lens is not an omni-directional lens, the processing means can combine individual images to produce and display high quality 360 degree images of the abdominal and pelvic cavities.

22. A system according to claim 19, wherein the processing means comprises memory means.

23. A system according to claim 19, wherein the wherein the observation unit is adapted to allow the operator to control one or more of the following parameters:
a. the camera frame rate;
b. the camera resolution;
c. the camera focus;
d. the camera zoom on selected regions of interest;
e. the level of illumination of the cavity;
f. the pressure and flow rate of gases; and
g. pressure in the abdominal and/or pelvic cavities.

24. A system according to claim 19, wherein some or all of the components of said system are controlled manually.

25. A system according to claim 19, wherein some or all of the components of said system are controlled automatically by means of software contained in the processing means.

26. A system according to claim 19, wherein the observation unit is adapted to allow the operator to select the display mode which allows him to present the imagery information gathered by the imaging device in the manner that is most clear to him.

27. A system according to claim 19, wherein the processing means of the observation unit enables stabilization of the acquired imagery information.

28. A system according to claim 19, wherein the observation unit is adapted to enable performance of medical image understanding (MIU) of the acquired information.

29. A system according to claim 28, wherein the MIU capabilities are used for automatic detection of abnormalities of tissues during surgery using an algorithm which enables analysis of the acquired images.

30. A system according to claim 28, wherein the MIU capabilities are used to automatically detect abnormalities during the surgery process.

31. A system according to claim 30, wherein the abnormalities are such as hemorrhages or operating tools accidentally left inside the cavity during surgery.

32. A system according to claim 19, wherein the observation unit is adapted to enable interface with additional medical imaging systems that might be present.

33. A system according to claim 32, wherein the interface enables three dimensional displays of the abdominal and pelvic cavities, based on all the information available from multiple sources.
